(19) United States
(12) Patent Application Publication
(10) Pub. No.: US 2009/0221811 A1
(43) Pub. Date: Sep. 3, 2009

(54) PROCESS FOR PREPARING GEMCITABINE AND ASSOCIATED INTERMEDIATES

(75) Inventors: Vladimir NADDAKA, Lod (IL); Eyal KLOPFER, Tel-Aviv (IL); Shady SAEED, Haifa (IL); Dionne MONTVILISKY, Givatayim (IL); Oded ARAD, Rehovot (IL); Joseph KASPI, Givatayim (IL)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: CHEMAGIS LTD., Bnei Brak (IL)

(21) Appl. No.: 12/430,525

(22) Filed: Apr. 27, 2009

Related U.S. Application Data
(62) Division of application No. 11/668,963, filed on Jan. 30, 2007.
(60) Provisional application No. 60/765,835, filed on Feb. 7, 2006.

(51) Int. Cl. C07H 19/06; C07D 317/30

(52) U.S. Cl. 536/28.5; 549/454

(57) ABSTRACT

The present invention provides processes for preparing intermediates useful in the preparation of gemcitabine and other nucleosides, and processes for preparing gemcitabine therewith. Exemplary intermediates include mixtures of D-erythro and D-threo isomers of 3-(hydroxy)-2,2-difluoro-3-(2,2-dimethyldioxolan-4-yl)-propionic acid salts. Also provided is a process for selectively isolating the D-erythro and D-threo isomers of D-erythro and D-threo isomers of 3-(hydroxy)-2,2-difluoro-3-(2,2-dimethyldioxolan-4-yl)-propionic acid salts, and processes for using such isomers in the preparation of nucleoside analogs such as, e.g., gemcitabine, intermediates thereof, and analogs thereof.

### CROSS-REFERENCE TO RELATED APPLICATIONS

This patent application is a divisional of U.S. patent application Ser. No. 11/668,963, filed Jan. 30, 2007, which claims the benefit of U.S. Provisional Patent Application No. 60/765,835, filed Feb. 7, 2006, all of which are incorporated by reference.

### BACKGROUND OF THE INVENTION

Gemcitabine HCl, marketed by Eli Lilly under the trademark Gemzar®, is a nucleoside analogue that exhibits antitumor activity and belongs to a general group of chemotherapy drugs known as antimetabolites. Gemcitabine prevents cells from producing DNA and RNA by interfering with the synthesis of nucleic acids, thus stopping the growth of cancer cells and causing them to die.

Gemcitabine is a synthetic glucoside analog of cytosine, which is chemically described as 4-amino-1-(2-deoxy-2,2-difluoro-β-D-ribofuranosyl)-pyrimidin-2(1H)-one or 2′-deoxy-2′,2′-difluorocytidine (β isomer). Gemcitabine HCl has the following structure:

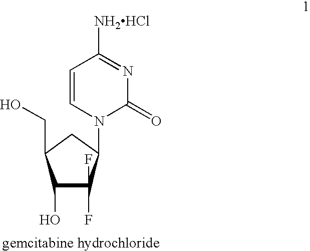

Gemzar® is supplied in vials as the hydrochloride salt in sterile form, only for intravenous use, containing either 200 mg or 1 g of gemcitabine HCl (as free base) formulated with mannitol (200 mg or 1 g, respectively) and sodium acetate (12.5 mg or 62.5 mg, respectively) as a sterile lyophilized powder. Hydrochloric acid and/or sodium hydroxide may have been added for pH adjustment.

U.S. Pat. No. 4,808,614 (“the '614 patent”) describes a process for synthetically producing gemcitabine, which process is generally illustrated in Scheme 1.

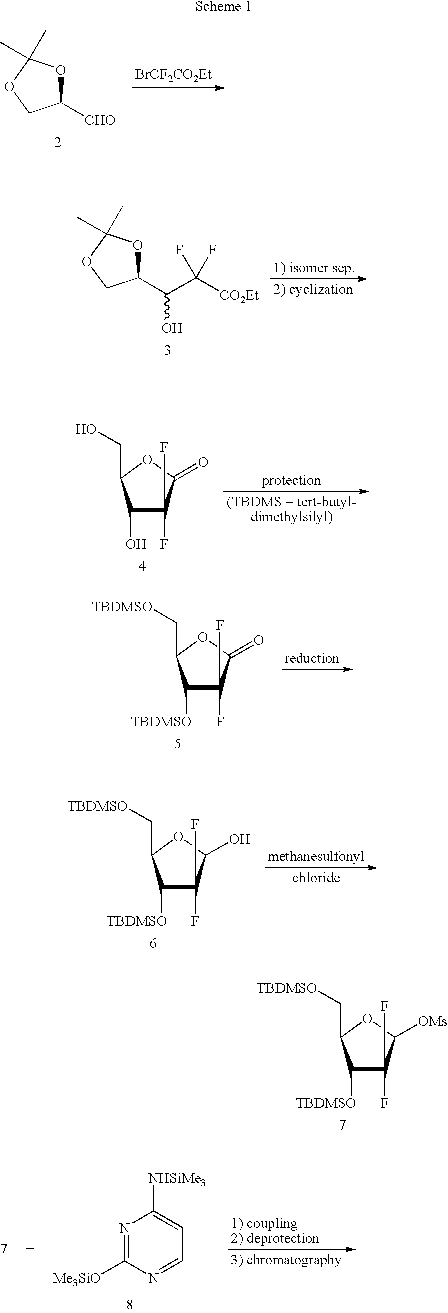

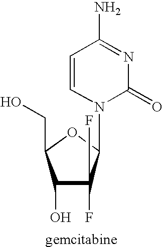

The D-glyceraldehyde ketal 2 is reacted with bromodifluoroacetic acid ethyl ester (BrCF₂COOEt) in the presence of activated zinc, to obtain ethyl 2,2-difluoro-3-hydroxy-3-(2,2-dimethyldioxolan-4-yl)-propionate 3 as a mixture of 3-R and 3-S isomers. The 3-R to 3-S isomer ratio is about 3:1. The 3-R isomer has the stereochemistry required for producing the desired erythro (3-R) ribose structure, and can be separated from the 3-S isomer by chromatography.

The resulting product is cyclized by treatment with an acidic ion exchange resin, such as Dowex 50W-X12, to produce 2-deoxy-2,2-difluoro-D-erythro-pentanoic acid-γ-lactone 4. The hydroxy groups of the lactone are protected with tert-butyldimethylsilyl (TBDMS) protecting groups to obtain the protected lactone 3,5-bis-(tert-butyldimethylsilyloxy)-2-desoxy-2,2-difluoro-1-oxoribose 5, and the product is reduced to obtain 3,5-bis-(tert-butyldimethylsilyl)-2-desoxy-2,2-difluororibose 6.

The 1-position of the carbohydrate is activated by the introduction of a leaving group, e.g., methanesulfonyloxy (mesylate), formed by reacting compound 6 with methanesulfonyl chloride to obtain 3,5-bis-(tert-butyldimethylsilyloxy)-1-methanesulfonyloxy-2-desoxy-2,2-difluororibose 7. The base ring is coupled to the carbohydrate by reacting compound 7 with N,O-bis-(trimethylsilyl)-cytosine 8 in the presence of a reaction initiator, such as trifluoromethanesulfonyloxy trimethylsilane (trimethylsilyl triflate). Removal of the protecting groups and chromatographic purification affords gemcitabine free base.

U.S. Pat. No. 4,526,988 describes a similar process in which the cyclization is carried out by hydrolyzing an alkyl 3-dioxolanyl-2,2-difluoro-3-hydroxy-propionate with a mildly acidic ion exchange resin. See also, Hertel et al., J. Org. Chem. 53, 2406 (1998).

U.S. Pat. No. 4,965,374 (“the '374 patent”) describes a process for producing gemcitabine from an intermediate 3,5-dibenzoyl ribo protected lactone of the formula:

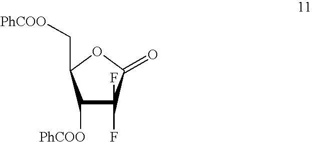

where the desired erythro isomer can be isolated in a crystalline form from a mixture of erythro and threo isomers. The process described in the '374 patent is generally outlined in Scheme 2.

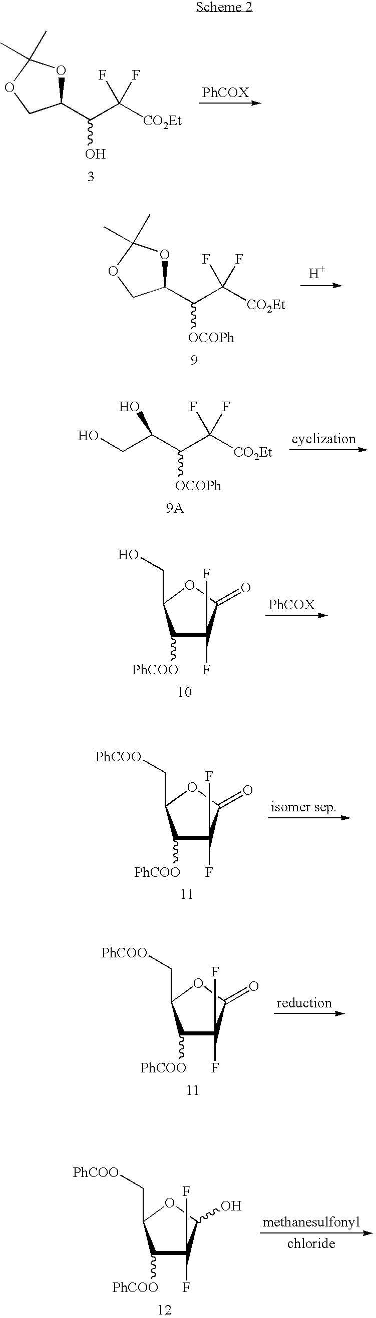

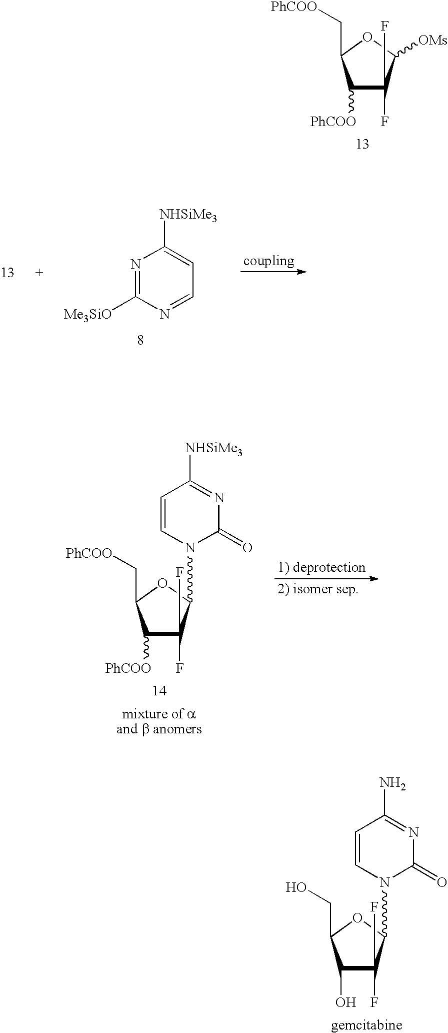

The 3-hydroxy group of compound 3 is esterified with a benzoyl protecting group by reaction with benzoyl chloride, benzoyl bromide, benzoyl cyanide, benzoyl azide, etc. (e.g., PhCOX, wherein X=Cl, Br, CN, or N₃), in presence of a tertiary amine or a catalyst such as 4-dimethylaminopyridine or 4-pyrrolidinopyridine, to obtain ethyl 2,2-difluoro-3-benzoyloxy-3-(2,2-dimethyldioxolan-4-yl)-propionate 9.

The isoalkylidene protecting group of 9 is selectively removed, e.g., by using a strong acid such as concentrated sulfuric acid in ethanol, to produce ethyl-2,2-difluoro-3-benzoyloxy-4,5-dihydroxypentanoate 9A. The product is cyclized to lactone 10 and converted to the dibenzoate ester to produce the lactone 2-deoxy-2,2-difluoropentofuranos-1-ulose-3,5-dibenzoate 11 as a mixture of erythro and threo isomers. The '374 patent describes isolating at least a portion of the erythro isomer from the mixture by selective precipitation. See also, Chou et al., Synthesis, 565-570, (1992).

Compound 11 is then reduced to obtain a mixture of α and β anomers of 2-desoxy-2,2-difluorpentofuranose-dibenzoate 12, which is activated with methane sulfonylchloride to obtain an anomeric mixture of mesylates, 2-deoxy-2,2-difluoro-D-ribofuranosyl-3,5-di-O-benzoyl-1-O-β-methanesulfonate 13, and coupled with N,O-bis(trimethylsilyl)-cytosine 8 to obtain silyl-protected nucleoside 14 as the dibenzoate ester as a mixture of the α- and β-anomers (about a 1:1 α/β anomer ratio). Removal of the esters and silyl protecting group provides a mixture of the β-anomer (gemcitabine) and the α-anomer (about a 1:1 α/β anomer ratio). The '374 patent describes selectively isolating the β-anomer (gemcitabine) by forming a salt of the anomeric mixture, e.g., the hydrochloride or hydrobromide salt, and selectively precipitating to obtain 2′-deoxy-2′,2′-difluorocytidine as the salt in 1:4 α/β ratio. The '374 patent also describes selectively precipitating the β-anomer in free base form in a slightly basic aqueous solution. One such process involves dissolving the 1:1 α/β anomeric mixture in hot acidic water (pH adjusted to 2.5-5.0) and, once the mixture is substantially dissolved, increasing the pH to 7.0-9.0 and allowing the solution to cool, to produce crystals, which are isolated by filtration.

Processes for separating anomeric mixtures of alkylsulfonate intermediates also have been described. U.S. Pat. Nos. 5,256,797 and 4,526,988 describe processes for separating anomers of 2-deoxy-2,2-difluoro-D-ribofuranosyl-1-alkylsulfonates, and U.S. Pat. No. 5,256,798 describes a process for obtaining α-anomer-enriched ribofuranosyl sulfonates.

Other intermediates that may be useful for preparing gemcitabine have been disclosed. For instance, U.S. Pat. No. 5,480,992 describes anomeric mixtures of 2,2-difluororibosyl azide and corresponding amine intermediates that can be prepared, e.g., by reacting a 2-deoxy-2,2-difluoro-D-ribofuranosyl-3,5-di-O-benzoyl-1-O-β-methanesulfonate with an azide nucleophile, such as lithium azide, to obtain the azide. Reduction of the azide produces the corresponding amine, which can be synthetically converted into a nucleoside. See also U.S. Pat. Nos. 5,541,345 and 5,594,155.

Other known intermediates include, e.g., tritylated intermediates (U.S. Pat. No. 5,559,222), 2-deoxy 2,2-difluoro-β-D-ribo-pentopyranose (U.S. Pat. No. 5,602,262), 2-substituted-3,3-difluorofuran intermediates (U.S. Pat. No. 5,633,367), and α,α-difluoro-β-hydroxy thiol esters (U.S. Pat. Nos. 5,756,775 and 5,912,366).

There are inherent problems associated with the production of gemcitabine, particularly for processes that require the production and separation of isomers, which tend to produce poor yields on a commercial scale. Accordingly, there is a need for improved methods of preparing gemcitabine and intermediates thereof, which facilitate the production of gemcitabine, particularly on a commercial scale. The present invention provides such methods and intermediates.

### BRIEF SUMMARY OF THE INVENTION

The present invention provides novel compounds which can be used as intermediates for the production of gemcitabine. In one aspect, the present invention provides a process for preparing a mixture of D-erythro and D-threo (3R- and 3S-) isomers of 3-(hydroxy)-2,2-difluoro-3-(2,2-dimethyldioxolan-4-yl)propionic acid salts, having the following structural formula 15:

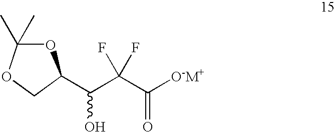

wherein M⁺ is Li⁺, Na⁺, K⁺, Ca²⁺, Ba²⁺ or R₁R₂R₃NH⁺, wherein R₁, R₂ and R₃ are the same or different and each is selected from hydrogen, saturated C₁-C₁₀ alkyl, saturated C₃-C₈ cycloalkyl, unsubstituted and substituted phenyl and unsubstituted and substituted heterocycloalkyl. The process preferably includes reacting a mixture of D-erythro and D-threo (3R- and 3S-) isomers of 2,2-difluoro-3-hydroxy-3-(2,2-dimethyldioxolan-4-yl)propionate esters of the formula 3A:

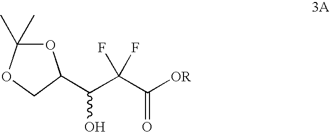

with a suitable base (e.g., an inorganic or organic base) in water, or in a water-miscible solvent, or in a mixture of water and a water-miscible solvent, wherein the inorganic base is lithium hydroxide, lithium bicarbonate, lithium carbonate, sodium hydroxide, sodium bicarbonate, potassium hydroxide, potassium bicarbonate, potassium carbonate, calcium oxide, calcium hydroxide, calcium carbonate, barium oxide, barium hydroxide, barium carbonate, or ammonium hydroxide and the organic base has the general formula R₁R₂R₃N, and R₁, R₂ and R₃ are as defined above. The R substituent in formula 3A can include any suitable substituent, e.g., which allows conversion of a compound of formula 3A into a compound of formula 15. Suitable R substituents in formula 3A can include, for example, substituted or unsubstituted alkyl (e.g., a substituted or an unsubstituted saturated C₁-C₁₀ alkyl, a substituted or an unsubstituted saturated C₁-C₆ alkyl, or a substituted or an unsubstituted saturated C₁-C₄ alkyl, e.g., ethyl), substituted or unsubstituted aryl (e.g., a substituted or an unsubstituted phenyl), and the like.

The present invention also provides a process for selectively isolating, in more than 95% purity, preferably more than 98.5% purity, and more preferably in a purity of more than 99% the D-erythro isomers of (3R)-3-(hydroxy)-2,2-difluoro-3-(2,2-dimethyldioxolan-4-yl)propionic acid salts of the general structural formula 15A:

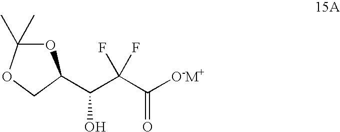

wherein M⁺ and R₁, R₂ and R₃ are as defined above, from a mixture of D-erythro and D-threo (3R- and 3S-) isomers of a compound of formula 15. The process preferably comprises dissolving the diastereomeric mixture of D-erythro and D-threo isomers in at least one solvent, optionally at elevated temperature, cooling the solution sufficiently to cause crystallization and collecting the precipitated D-erythro isomer.

The present invention additionally provides a high-yield process for preparing the compound having the structural formula 4:

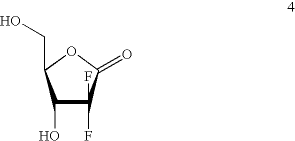

which preferably includes hydrolyzing a D-erythro isomer of formula 15A using an acid as a hydrolytic reagent, followed by removal of water preferably by azeotropic distillation.

The present invention further provides a process for selective isolating, in more than 96.5% purity, preferably in more than 99% purity, and more preferably in a purity equal to or greater than 99.7%, the D-threo isomer (3S)-3-hydroxy-2,2-difluoro-3-(2,2-dimethyldioxolan-4-yl)propionic acid dicyclohexylammonium salt having the structural formula 15B:

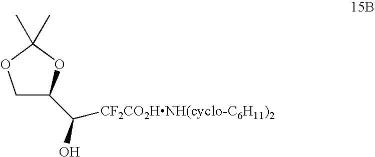

from the filtrate solution obtained after selective precipitation of the D-erythro isomer, which process preferably includes:

optionally reducing the volume of the filtrate solution by evaporation;

allowing the crystals of the D-threo isomer to precipitate;

collecting the crystals by filtration; and

optionally washing the obtained crystals, and drying.

The present invention further provides a process for preparing the xylo lactone, having the structural formula 4B:

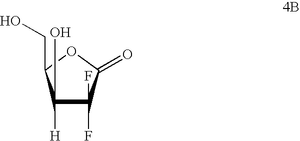

the process comprising hydrolyzing a D-threo isomer of formula 15B using an acid as a hydrolytic reagent, followed by removal of water preferably by azeotropic distillation.

The present invention further provides a process for preparing 3,5-disubstituted-2-deoxy-2,2-difluoro-D-erythro-pentofuranos-1-uloses, having the structural formula 11A, and 3,5-disubstituted-2-deoxy-2,2-difluoro-D-threo-pentofuranos-1-uloses, having the formula 11B:

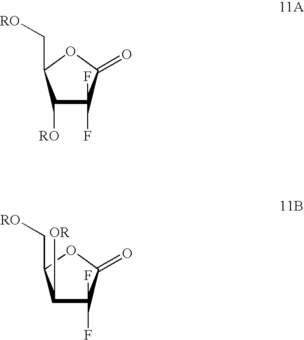

wherein R is any suitable substituent, including but not limited to phenyl, 2-phenylethenyl (to form the cinnamoyl ester), 1-naphthylmethyl, 2-methylbenzyl, 4-methylbenzyl or the like, the process comprising reacting the ribo lactone 4 or the xylo lactone 4B with a silylating or acylating reagent.

### DETAILED DESCRIPTION OF THE INVENTION

It has been surprisingly discovered that mixtures of D-erythro and D-threo (3R- and 3S-) isomers of 3-(hydroxy)-2,2-difluoro-3-(2,2-dimethyldioxolan-4-yl)propionic acid salts may be readily separated to obtain, e.g., the D-erythro isomer in a purity greater than 95%, preferably in a purity greater than 98.5%, and more preferably in a purity greater than 99%. In accordance with the present invention, such diastereomeric mixtures can be utilized as intermediates for the production of gemcitabine.

In one aspect, the present invention provides a novel process for preparing a mixture of D-erythro and D-threo (3R- and 3S-) isomers of 3-(hydroxy)-2,2-difluoro-3-(2,2-dimethyldioxolan-4-yl)propionic acid salt, having the following structural formula 15:

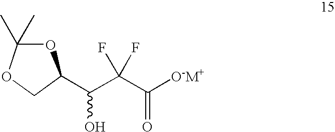

wherein M⁺ is Li⁺, Na⁺, K⁺, Ca²⁺, Ba²⁺ or R₁R₂R₃NH⁺, wherein R₁, R₂ and R₃ are the same or different and each is selected from hydrogen, saturated C₁-C₁₀ alkyl, saturated C₃-C₈ cycloalkyl, unsubstituted and substituted phenyl and unsubstituted and substituted heterocycloalkyl; which process preferably includes reacting a mixture of D-erythro and D-threo (3R- and 3S-) isomers of 2,2-difluoro-3-hydroxy-3-(2,2-dimethyldioxolan-4-yl)propionate esters of formula 3A:

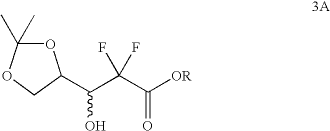

which can be suspended in water, or in a water-miscible solvent, or in a mixture of water and a water-miscible solvent, with a base, e.g., an inorganic or organic base, wherein the inorganic base is lithium hydroxide, lithium bicarbonate, lithium carbonate, sodium hydroxide, sodium bicarbonate, potassium hydroxide, potassium bicarbonate, potassium carbonate, calcium oxide, calcium hydroxide, calcium carbonate, barium oxide, barium hydroxide, barium carbonate, or ammonium hydroxide and the organic base has the general formula R₁R₂R₃N, and R₁, R₂ and R₃ are as defined herein. The R substituent in formula 3A can include any suitable substituent, e.g., which allows conversion of a compound of formula 3A into a compound of formula 15. Suitable R substituents in formula 3A can include, for example, substituted or unsubstituted alkyl (e.g., a substituted or an unsubstituted saturated C₁-C₁₀ alkyl, a substituted or an unsubstituted saturated C₁-C₆ alkyl, or a substituted or an unsubstituted saturated C₁-C₄ alkyl, e.g., ethyl), substituted or unsubstituted aryl (e.g., a substituted or an unsubstituted phenyl), and the like.

In another embodiment, the present invention provides a process for preparing the mixture of D-erythro and D-threo (3R- and 3S-) isomers of 3-(hydroxy)-2,2-difluoro-3-(2,2-dimethyldioxolan-4-yl)propionic acid salt, which process includes:

reacting the compound of formula 3A with a suitable base suspended in water or in a water-miscible solvent, or in a mixture of water and a water-miscible solvent, optionally at elevated temperature;

allowing the reaction mixture to cool sufficiently and isolating the thus formed precipitate by filtration;

optionally slurrying the solid precipitate in an organic solvent, optionally at elevated temperature; and

collecting the product, e.g., by filtration, and optionally washing and/or drying.

Suitable bases can include organic bases and inorganic bases. Preferably, the base is an organic base (e.g., an organic amine) selected from n-butylamine, sec-butylamine, isobutylamine, n-pentylamine, n-hexylamine, cyclohexylamine, cycloheptylamine, dipropylamine, diisopropylamine, dibutylamine, diisobutylamine, dicyclohexylamine, piperidine, 2,6-dimethylpiperidine, 4-(dimethylamino)pyridine, benzylamine, and the like, and combinations thereof. Preferred organic bases include cyclohexylamine, dicyclohexylamine, 4-(dimethylamino)pyridine, benzylamine, and the like, and combinations thereof. Preferred inorganic bases include sodium hydroxide, sodium bicarbonate, sodium carbonate, and the like, and combinations thereof.

Exemplary water-miscible solvents can include, e.g., one or more solvents selected from methanol, ethanol, 1-propanol, 2-propanol, acetonitrile, acetone, N,N-dimethylformamide (DMF), N,N-dimethylacetamide (DMA), dimethyl sulfoxide (DMSO), and the like, and combinations thereof. Preferred solvents include acetonitrile and 2-propanol.

Exemplary solvents for slurrying the obtained precipitate can include, e.g., one or more solvents selected from diethyl ether, diisopropyl ether, tert-butyl methyl ether, methyl acetate, ethyl acetate, n-propyl acetate, isopropyl acetate, n-butyl acetate, isobutyl acetate, hexane, heptane, cyclohexane, petrol ether, and the like, and combinations thereof. Preferred solvents for slurrying the obtained precipitate include tert-butyl methyl ether and ethyl acetate.

Exemplary salts of D-erythro isomers of the (3R)-3-(hydroxy)-2,2-difluoro-3-(2,2-dimethyldioxolan-4-yl)propionic acid include compounds having the general structural formula 15A:

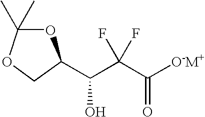

wherein M⁺ is Li⁺, Na⁺, K⁺, Ca²⁺, Ba²⁺ or R₁R₂R₃NH⁺, wherein R₁, R₂ and R₃ are as defined herein, including, e.g., ammonium salt, n-butyl ammonium salt, sec-butyl ammonium salt, isobutyl ammonium salt, n-pentyl ammonium salt, n-hexyl ammonium salt, cyclohexyl ammonium salt, cycloheptyl ammonium salt, dipropyl ammonium salt, diisopropyl ammonium salt, dibutyl ammonium salt, diisobutyl ammonium salt, dicyclohexyl ammonium salt, morpholinium salt, piperidinium salt, 2,6-dimethylpiperidinium salt, 4-(dimethylamino)pyridinium salt, benzyl ammonium salt, and the like.

The present invention also provides a novel process for separating the mixture of D-erythro and D-threo (3R- and 3S-) isomers of 3-(hydroxy)-2,2-difluoro-3-(2,2-dimethyldioxolan-4-yl)propionic acid salts, to obtain the D-erythro isomers of the general structural formula 15A thereof. Preferably, the separation process includes:

suspending the mixture of D-erythro and D-threo (3R- and 3S-) of 3-(hydroxy)-2,2-difluoro-3-(2,2-dimethyldioxolan-4-yl)propionic acid salt in at least one solvent, optionally at elevated temperature;

cooling the mixture and setting aside for sufficient time to allow crystallization; and

collecting the thus formed crystals, e.g., by filtration, and optionally washing and/or drying.

Exemplary solvents used for suspending the mixture of the two isomers D-erythro and D-threo (3R- and 3S-) of 3-(hydroxy)-2,2-difluoro-3-(2,2-dimethyl-dioxolan-4-yl)propionic acid salt include methyl acetate, ethyl acetate, n-propyl acetate, isopropyl acetate, n-butyl acetate, isobutyl acetate, methanol, ethanol, 1-propanol, 2-propanol, acetonitrile, acetone, water, and the like, and mixtures thereof. Preferred solvents include mixtures of acetonitrile and water, mixtures of 2-propanol and ethyl acetate, and the like, and combinations thereof.

The process of the present invention can produce D-erythro isomers (3R)-3-(hydroxy)-2,2-difluoro-3-(2,2-dimethyldioxolan-4-yl)propionic acid salts of the general structural formula 15A in purity greater than 95%, preferably in a purity greater than 98.5%, and more preferably in a purity greater than 99%.

In yet another embodiment, the present invention provides high-yield processes for preparing the compound 2-deoxy-2,2-difluoro-D-erythro-pentofuranose-1-ulose of the structural formula 4:

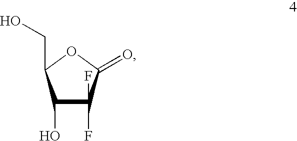

which is valuable as a precursor in the synthesis of gemcitabine, by hydrolyzing the D-erythro isomer of formula 15A using an acid as a hydrolytic reagent, followed by removal of water preferably by azeotropic distillation. The process preferably includes:

combining the D-erythro isomer of formula 15A and a mixture comprising a water-miscible solvent, water and an acid;

heating the mixture for a sufficient time period to allow completion of the reaction;

optionally reducing the solution volume by distillation;

adding a water immiscible solvent and removing the water, preferably by azeotropic distillation;

further distilling off the solvent mixture to obtain the product;

adding an organic solvent, and precipitating an ammonium salt by-product; and

evaporating the solvent to obtain the product.

Exemplary water-miscible solvents include acetonitrile, tetrahydrofuran (THF), 2-methyltetrahydrofuran, acetone, N,N-dimethylformamide (DMF), N,N-dimethylacetamide (DMA), and mixtures thereof. A preferred water-miscible solvent is acetonitrile.

Exemplary acids include methanesulfonic acid, sulfuric acid, trifluoroacetic acid, and the like, and combinations thereof. Preferred acids include trifluoroacetic acid, sulfuric acid, and combinations thereof.

Exemplary mixtures of water-miscible solvent, water and an acid include mixtures of acetonitrile, water and trifluoroacetic acid, preferably in acetonitrile:water:trifluoroacetic acid ratios of 100:5:2.8 v/v/v or 100:10:2.0 v/v/v, and mixtures of acetonitrile, water and sulfuric acid, in exemplary acetonitrile:water:sulfuric acid, preferably in ratios of 100:5:1.2 v/v/v or 100:10:1.2 v/v/v.

Exemplary water-immiscible solvents include toluene, o-xylene, m-xylene, p-xylene, diethylbenzene, and the like, and mixtures thereof. A preferred water-immiscible solvent is toluene.

Exemplary solvents used for precipitating the ammonium salt by-product and for obtaining the product upon evaporation include diethyl ether, diisopropyl ether, tert-butylmethyl ether, methyl acetate, ethyl acetate, n-propyl acetate, isopropyl acetate, n-butyl acetate, isobutyl acetate, and the like, and mixtures thereof, of which diethyl ether and ethyl acetate are preferred.

The azeotropic distillation of water can be carried out using a Dean-Stark trap, e.g., to dry the toluene, e.g., by heating the mixture at reflux.

The experimental results of preparing the compound of formula 4 are summarized in the following table:

| | | | Reagent | Precipitating | Product's | Product's |
|---|---|---|---|---|---|---|
| Process/example | Acid | Reagent mixture | combination | solvent | yield % | purity % |
| Process A/ | TFA | acetonitrile:water:TFA | 100:5:2.8 | diethyl ether | 99.9 | 93.2 |
| example 9 | | | | | | |
| Process B/ | TFA | acetonitrile:water:TFA | 100:10:2.0 | ethyl acetate | 99.9 | 88.1 |
| example 10 | | | | | | |
| Process C/ | H₂SO₄ | acetonitrile:water:H₂SO₄ | 100:5:1.2 | diethyl ether | 99.9 | 87.6 |
| example 11 | | | | | | |
| Process D/ | H₂SO₄ | acetonitrile:water:H₂SO₄ | 100:10:1.2 | ethyl acetate | 90.5 | 93.4 |
| example 12 | | | | | | |
| Process E/ | H₂SO₄ | acetonitrile:water:H₂SO₄ | 100:5:1.2 | ethyl acetate | 82.5 | 98.7 |
| example 13 | | | | | | |

In accordance with the present invention, 2-deoxy-2,2-difluoro-D-erythro-pentofuranose-1-ulose of formula 4 can be obtained in high yields, e.g., in a yield of at least about 95%, preferably in a yield of at least about 99%, and more preferably in a yield of at least about 99.9%.

The present invention further provides a process for selectively isolating, in more than 96.5% purity, preferably in more than 99% purity, and more preferably in a purity equal to or greater than 99.7%, the D-threo isomer (3S)-3-hydroxy-2,2-difluoro-3-(2,2-dimethyldioxolan-4-yl)propionic acid dicyclohexylammonium salt having the structural formula 15B:

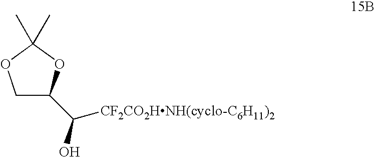

from the filtrate solution obtained after selective precipitation of the D-erythro isomer. The isolation process preferably comprises:

optionally reducing the volume of the filtrate solution by evaporation;

allowing the crystals of the D-threo isomer to precipitate;

collecting the crystals by filtration; and

optionally washing and/or drying the obtained crystals.

Exemplary solvents used for washing the obtained crystals include acetonitrile, methyl acetate, ethyl acetate, n-propyl acetate, isopropyl acetate, n-butyl acetate, isobutyl acetate, acetone, and the like, and mixtures thereof. A preferred solvent for washing the crystals is acetonitrile.

In yet another embodiment, the present invention provides a process for preparing the xylo lactone, 2-deoxy-2,2-difluoro-D-thero-pentofuranose-1-ulose having the structural formula 4B:

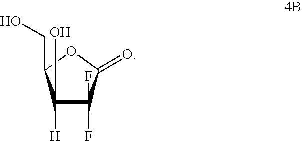

The process preferably includes hydrolyzing a D-threo isomer of formula 15B using an acid as a hydrolytic reagent, followed by removal of water preferably by azeotropic distillation.

In yet another embodiment, the present invention provides a process for preparing 3,5-disubstituted-2-deoxy-2,2-difluoro-D-erythro-pentofuranos-1-uloses, having the structural formula 11A, and 3,5-disubstituted-2-deoxy-2,2-difluoro-D-threo-pentofuranos-1-uloses, having the formula 11B:

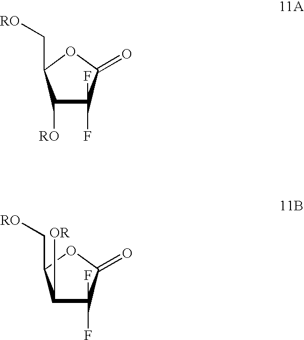

wherein R is any suitable substituent, including but not limited to acetyl, phenyl, 2-phenylethenyl (to form the cinnamoyl ester), phenyl acetyl, 1-naphthylmethyl, benzyl, 2-methylbenzyl, 4-methylbenzyl, 2-chlorobenzyl, 4-chlorobenzyl, and the like, the process preferably comprises reacting the ribo lactone 11A or the xylo lactone 11B with a silylating or acylating reagent.

An exemplary process for preparing the 3,5-disubstituted-2-deoxy-2,2-difluoro-D-erythro-pentofuranos-1-uloses, having the structural formula 11A, includes:

dissolving 2-deoxy-2,2-difluoro-D-erythro-pentofuranos-1-ulose having the structural 4 in an organic solvent, optionally under inert atmosphere;

adding at least one base and an acid chloride, optionally drop-wise;

refluxing the mixture for sufficient time period to allow completion of the reaction;

filtering out the ammonium salt by-product and cooling the filtrate to obtain a second portion, and evaporating the solvent to obtain a solid; and

optionally purifying the product.

Exemplary solvents used in the process are selected from the group consisting of diethyl ether, diisopropyl ether, t-butylmethyl ether, methyl acetate, ethyl acetate, n-propyl acetate, isopropyl acetate, n-butyl acetate, isobutyl acetate, and the like, and mixtures thereof. A preferred solvent is ethyl acetate.

In accordance with the present invention, suitable acid chlorides, which are useful for esterifying the hydroxy groups, include one or more acid chlorides selected from a group consisting of cinnamoyl chloride, 1-naphthoyl chloride, 1-naphthyl acetyl chloride, 4-methylphenyl chloride, acetyl chloride, phenyl chloride, acetyl chloride, 1-naphthylmethyl chloride, benzyl chloride, 2-methylbenzyl chloride, 4-methylbenzyl chloride, 2-chlorobenzyl chloride, 4-chlorobenzyl chloride and the like.

The base can include inorganic and organic bases, although the base used in the reaction is preferably at least one organic base selected from triethyl amine, lutidines, diisopropylethylamine, pyridine, 2-(dimethylamino)pyridine, 4-(dimethylamino)pyridine and the like. In a preferred embodiment, the base is an organic base which is a mixture of pyridine and 4-(dimethylamino)pyridine.

In accordance with the present invention, the compound of structural formula 11A or 11B can be purified by conventional methods known in the art including, without limitation, precipitation, crystallization, slurrying, washing in a suitable solvent, filtration through a packed-bed column, dissolution in an appropriate solvent (e.g., ethyl acetate) and re-precipitation by addition of a second solvent in which the compound is insoluble, or any combination of such methods.

According to the present invention, suitable solvents for slurrying and/or washing the compound of structural formula 11A or 11B include diethyl ether, diisopropyl ether, t-butylmethyl ether, methyl acetate, ethyl acetate, n-propyl acetate, isopropyl acetate, n-butyl acetate, isobutyl acetate, toluene, pentane, hexane, heptane, cyclohexane, petrol ether, and the like, and mixtures thereof, preferably toluene, ethyl acetate, or a mixture of ethyl acetate and hexane.

In accordance with the present invention, the compound of structural formula 11A or 11B can be obtained in high yields, e.g., in a yield of about 94%, having a purity of at least about 98.2%, and preferably a purity of at least about 99% and more preferably a purity of at least 99.7%

Scheme 3 outlines the process of the present invention for preparing gemcitabine or salts thereof, starting from a mixture of D-erythro and D-threo (3R- and 3S-) isomers of 2,2-difluoro-3-hydroxy-3-(2,2-dimethyldioxolan-4-yl)propionate esters of formula 3A:

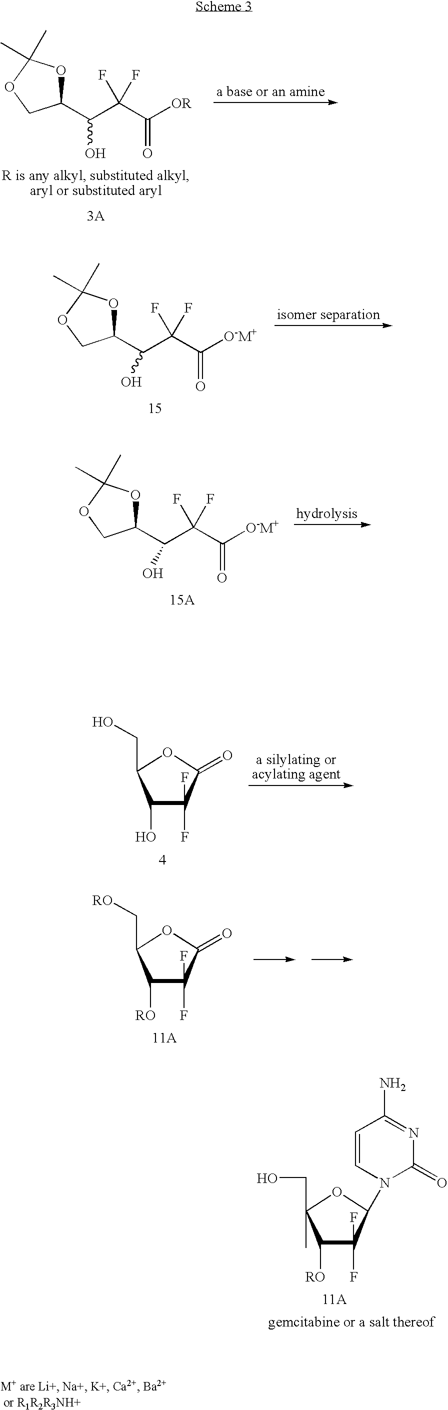

The following examples further illustrate the invention but, of course, should not be construed as in any way limiting its scope.

### Example 1

This example describes the preparation of D-erythro-3-(hydroxy)-2,2-difluoro-3-(2,2-dimethyldioxolan-4-yl)propionic acid dicyclohexylammonium salt (Method A).

An isomeric mixture of ethyl (D-erythro, D-threo)-3-(hydroxy)-2,2-difluoro-3-(2,2-dimethyldioxalane-4-yl)propionate [having a purity of 88.7% (by HPLC) and a ratio of 2.78 to 1 between the D-erythro-isomer and the D-threo-isomer; 50.0 g, 0.175 mol] and dicyclohexylamine (45.5 g, 50.0 ml, 0.248 mol, 1.4 equiv. with respect to the isomeric mixture of the D-erythro and D-threo) in water (250 ml) was heated to 80-95° C. for about one hour to obtain a suspension. The suspension was kept at ambient temperature for 3 hours during which time a crystalline solid was formed, which was collected by filtration and slurried in tert-butyl methyl ether (MTBE) (100 ml) at reflux temperature for one hour. The mixture was cooled to ambient temperature. The crystalline product was collected by filtration, washed with MTBE and dried at 50° C. overnight to yield 39.8 g of D-erythro-3-(hydroxy)-2,2-difluoro-3-(2,2-dimethyldioxolan-4-yl)propionic acid dicyclohexyl-ammonium salt in 55.9% yield (76.0% calculated from D-erythro diastereomer); having a purity of 97.34% by HPLC (containing 1.63% of D-threo diastereomer). The remaining solvent mixture was partially removed under reduced pressure and the residue was cooled at 5° C. for 2 hours. The colorless crystals were collected by filtration, washed with cold acetonitrile and dried at 50° C. overnight to give an additional amount of 3.8 g of the salt having a purity of 95.9% by HPLC (containing 0.67% of D-threo diastereomer).

The salt was dissolved in a mixture of acetonitrile (320 ml) and water (32 ml) under reflux. The solution was cooled to ambient temperature and kept at this temperature overnight. The colorless crystals were collected by filtration, washed with cold acetonitrile and dried at 50° C. overnight to give 36.6 g of the salt having a purity of 99.2% by HPLC, (containing 0.09% of D-threo diastereomer) in 51.4% yield, (69.9% calculated from the D-erythro diastereomer); mp 210-212° C., [α]_{D}²⁵+14.6° [c 1, acetonitrile (92%)-water (8%)]. ¹H NMR (CDCl₃): δ=1.22-2.08 (m, 20H, CH₂ in cyclohexyl), 1.40, 1.47 [d, 6H, C(CH₃)₂], 3.08 (m, 2H, CH in cyclohexyl), 4.16 (m, 2H, 5-CH₂ and 1H, 4-CH), 4.38 (q, 1H, 3-CH), 4.74 (s, 1H, 3-OH), 8.88 (s, 2H, ⁺NH₂cyclohexyl₂). ¹³C NMR (CDCl₃): δ=24.8, 25.1 and 29.1 (CH₂ in cyclohexyl), 25.6, 26.5 [C(CH₃)₂], 53.4 (CH in cyclohexyl), 65.4 (C-5, J_{C—F}=5 Hz), 71.8 (C-3, J_{C—F}=21.8, 25.8 Hz), 74.3 (C-4), 109.1 [C(CH₃)₂], 114.4 (C-2, J_{C—F}=252, 252 Hz), 167.9 (C-1, J_{C—F}=30, 30 Hz). The ¹⁹F NMR indicated that only one product containing fluorine was present.

### Example 2

This example describes the preparation of D-threo-3-(hydroxy)-2,2-difluoro-3-(2,2-dimethyldioxolan-4-yl)propionic acid dicyclohexylammonium salt (Method A).

The filtrate solution of example 1 was set aside for 24 hours, after which time the crystals of the D-threo isomer were precipitated. The crystals were collected by filtration, washed with acetonitrile and dried at 50° C. overnight to give 3.8 g of crude (D-threo)-3-(2,2-dimethyldioxolan-4-yl)propionic acid dicyclohexylammonium salt 15B, having a purity of 95.4%, (by HPLC), and containing 2.68% of the D-erythro diastereomer. The crude D-threo isomer was re-crystallized from acetonitrile to yield 3 g of the product, having a purity of 99.8% (by HPLC) and containing 0.13% of the D-erythro diastereomer, mp 184.0-187.0° C., [α]_{D}²⁵−14.60 [c 1.01, acetonitrile (92%)-water (8%)].

### Example 3

This example describes the preparation of D-erythro-3-(hydroxy)-3-2,2-difluoro-(2,2-dimethyldioxolan-4-yl)propionic acid dicyclohexylammonium salt (Method B).

An isomeric mixture of ethyl (D-erythro, D-threo)-3-(hydroxy)-2,2-difluoro-3-(2,2-dimethyldioxalane-4-yl)propionate [having a purity of 88.7% (by HPLC) and a ratio of 2.78 to 1 between the D-erythro-isomer to the D-threo-isomer, 10.0 g, 0.035 mol] and dicyclohexylamine (9.1 g, 5.0 ml, 0.050 mol, 1.4 equiv.) was suspended in a mixture of acetonitrile:water (10:1, 88 ml) and heated under reflux for about one hour to obtain a solution. The solution was cooled to ambient temperature and kept at this temperature for 3 hours. The colorless crystals were collected by filtration, washed with acetonitrile (10 ml) and dried at 50° C. overnight to give 8.1 g of D-erythro-3-(hydroxy)-2,2-difluoro-3-(2,2-dimethyldioxolan-4-yl)propionic acid dicyclohexylammonium salt in 56.8% yield, (77.2% yield calculated from D-erythro diastereomer); having a purity of 97.6% containing 1.25% of D-threo diastereomer (by HPLC).

### Example 4

This example describes the preparation of D-threo-3-(hydroxy)-3-2,2-difluoro-(2,2-dimethyldioxolan-4-yl)propionic acid dicyclohexylammonium salt (Method B).

The filtrate of example 3 was concentrated to about ⅔ of its volume and cooled to ambient temperature. The crystals were collected by filtration after one hour, washed with acetonitrile and dried at 50° C. overnight to obtained 2.0 g of the crude (D-threo)-3-(2,2-dimethyldioxolan-4-yl)propionic acid dicyclohexylammonium salt 15B, having a purity of 96.5% (by HPLC), containing 2.73% of D-erythro diastereomer.

### Example 5

This example describes the preparation of D-erythro-3-(hydroxy)-2,2-difluoro-3-(2,2-dimethyldioxolan-4-yl)propionic acid dimethyl(4-pyridyl)ammonium salt.

An isomeric mixture of ethyl (D-erythro, D-threo)-3-(hydroxy)-2,2-difluoro-3-(2,2-dimethyldioxalane-4-yl)propionate [having a purity of 88.7% (by HPLC) and a ratio of 2.78 to 1 between the D-erythro-isomer and the D-threo-isomer, 5.0 g, 0.0175 mol] and (4-dimethylamino)pyridine (2.35 g, 0.019 mol, 1.1 equiv) was suspended in a mixture of 2-propanol:water (1:1, 100 ml) and heated under reflux for 6 hours to obtain a solution. The solvents were removed to dryness under reduced pressure to obtain an oil. Ethyl acetate (10 ml) and 2-propanol (10 ml) were added to the oil and the mixture was slurried at ambient temperature for 4 hours. The colorless crystals were collected by filtration and dried at 50° C. overnight to yield 2.2 g of D-erythro-3-(hydroxy)-2,2-difluoro-3-(2,2-dimethyldioxolan-4-yl)propionic acid dimethyl(4-pyridyl)ammonium salt in 36.1% yield, having a purity of 96.2% containing 5.2% of D-threo diastereomer (by HPLC). The salt (2.2 g) was heated under reflux in a mixture of 2-propanol:ethyl acetate (2:1, 18 ml) to obtain a solution. The solution was cooled to ambient temperature and kept at this temperature for 3 hours. The colorless crystals were collected by filtration and dried at 50° C. overnight to give 1.8 g of the final product in 29.5% yield (40.1% calculated from the D-erythro diastereomer); having a purity of 98.2% containing 1.1% of the D-threo diastereomer (by HPLC); mp 166-168° C., [α]_{D}²⁵+8.9° (c 1, acetonitrile). ¹H NMR (D₂O): δ=1.36, 1.43 [d, 6H, C(CH₃)₂], 3.18 [s, 6H, N(CH₃)₂], 4.03-4.36 (m, 2H, 5-CH₂, 1H, 4-CH and 1H, 3-CH), 4.84 [s, 2H, 3-OH and HN⁺(CH₃)₂C₅H₄N], 6.84, 6.86 (d, 2H, 4-pyridyl), 8.00, 8.03 (d, 2H, 4-pyridyl). ¹³C NMR (D₂O): δ=24.4, 25.4 [C(CH₃)₂], 53.4 [N(CH₃)₂], 64.8 (C-5), 70.6 (C-3, J_{C—F}=21.8, 25.8 Hz), 73.8 (C-4, J_{C—F}=5 Hz), 109.9 [C(CH₃)₂], 115.9 (C-2, J_{C—F}=252, 252 Hz), 106.8, 138.3 and 157.4 (C₅H₄N), 168.7 (C-1, J_{C—F}=30, 30 Hz). The ¹⁹F NMR indicated that only one product containing fluorine was present.

### Example 6

This example describes the preparation of D-erythro-3-(hydroxy)-2,2-difluoro-3-(2,2-dimethyldioxolan-4-yl)propionic acid cyclohexylammonium salt.

An isomeric mixture of ethyl (D-erythro, D-threo)-3-(hydroxy)-2,2-difluoro-3-(2,2-dimethyldioxalane-4-yl)propionate [having a purity of 88.7% (by HPLC) and a ratio of 2.78 to 1 between the D-erythro-isomer and the D-threo-isomer, 10.0 g, 0.035 mol] and cyclohexylamine (5.2 g, 6.0 ml, 0.052 mol, 1.5 equiv) was suspended in a mixture of acetonitrile:water (10:1, 88 ml) and heated under reflux for about one hour to obtain a solution. The solution was cooled to ambient temperature and kept at this temperature for 4 hours. The colorless crystals were collected by filtration, washed with acetonitrile (10 ml) and dried at 50° C. overnight to give 5.3 g of D-erythro-3-(hydroxy)-2,2-difluoro-3-(2,2-dimethyldioxolan-4-yl)propionic acid cyclohexyl-ammonium salt in 46.5% yield, (63.2% calculated from the D-erythro diastereomer); having a purity of 98.6% (by HPLC) containing 1.4% of D-threo diastereomer; mp 209-211° C., [α]_{D}²⁵+17.5° [c 1, acetonitrile (92%)-water (8%)]. ¹H NMR (D₂O): δ=1.08-2.01 (m, 10H, CH₂ in cyclohexyl), 1.38, 1.44 [d, 6H, C(CH₃)₂], 3.15 (m, 2H, CH in cyclohexyl), 4.04-4.26 (m, 2H, 5-CH₂ and 1H, 4-CH), 4.35 (q, 1H, 3-CH), 4.80 (s, 4H, 3-OH and ⁺NH₃cyclohexyl). ¹³C NMR (D₂O): δ=24.5, 24.9 and 31.0 (CH₂ in cyclohexyl), 24.8, 25.9 [C(CH₃)₂], 51.0 (CH in cyclohexyl), 65.4 (C-5), 71.2 (C-3, J_{C—F}=21.8, 25.8 Hz), 74.3 (C-4, J_{C—F}=5 Hz), 109.1 [C(CH₃)₂], 116.4 (C-2, J_{C—F}=252, 252 Hz), 169.6 (C-1, J_{C—F}=25, 25 Hz). The ¹⁹F NMR indicated that only one product containing fluorine was present.

### Example 7

This example describes the preparation of D-erythro-3-(hydroxy)-2,2-difluoro-3-(2,2-dimethyldioxolan-4-yl)propionic acid benzylammonium salt.

An isomeric mixture of ethyl (D-erythro, D-threo)-3-(hydroxy)-2,2-difluoro-3-(2,2-dimethyldioxalane-4-yl)propionate [having a purity of 88.7% (by HPLC) and a ratio of 2.78 to 1 between the D-erythro-isomer to the D-threo-isomer; 5.0 g, 0.0175 mol] and benzylamine (2.8 g, 2.9 ml, 0.026 mol, 1.5 equiv) was suspended in a mixture of acetonitrile:water (10:1, 40 ml) and heated under reflux for about one hour to obtain a solution. The solution was cooled to ambient temperature and kept at this temperature for 4 hours. The colorless crystals were collected by filtration, washed with acetonitrile (5 ml) and dried at 50° C. overnight to give 2.8 g of D-erythro-3-(hydroxy)-2,2-difluoro-3-(2,2-dimethyldioxolan-4-yl)propionic acid benzylammonium salt in 48.0% yield (calculated as 65.2% from the D-erythro diastereomer); having a purity of 97.6% containing 1.5% of D-threo diastereomer (by HPLC); mp 160-162° C., [α]_{D}²⁵+14.8° [c 1, acetonitrile (92%)-water (8%)]. ¹H NMR (D₂O): δ=1.36, 1.43 [d, 6H, C(CH₃)₂], 4.03-4.26 (m, 2H, 5-CH₂ and 1H, 4-CH), 4.18 (s, 2H, CH₂ in CH₂Ph), 4.33 (q, 1H, 3-CH), 4.79 (s, 4H, 3-OH and ⁺NH₃-benzyl), 7.47 (m, 5Hₐᵣₒₘ). ¹³C NMR (D₂O): δ=24.8, 25.9 [C(CH₃)₂], 43.8 (CH₂ in benzyl), 65.4 (C-5), 71.2 (C-3, J_{C—F}=21.8, 25.8 Hz), 74.3 (C-4, J_{C—F}=5 Hz), 110.6 [C(CH₃)₂], 116.4 (C-2, J_{C—F}=252, 252 Hz), 129.5, 129.9 and 133.2 (Cₐᵣₒₘ), 169.6 (C-1, J_{C—F}=25, 25 Hz). The ¹⁹F NMR indicated that only one product fluorine containing was present.

### Example 8

This example describes the preparation of D-erythro-3-(hydroxy)-2,2-difluoro-3-(2,2-dimethyldioxolan-4-yl)propionic acid sodium salt.

A mixture of ethyl (D-erythro, D-threo)-3-(hydroxy)-2,2-difluoro-3-(2,2-dimethyldioxolan-4-yl)propionate (IV) (94.0% purity, a ratio R to S is 3 to 1, 10.0 g, 0.037 mol) and 46% aqueous NaOH solution (3.5 g, 0.040 mol, 1.1 eq.) in water (30 ml) was heated at 80° C. for half hour to obtain an yellow solution. Then, the water was removed from the solution under reduced pressure to obtain brownish oil. The oil was dissolved at heating in an ethyl acetate-diethyl ether (3:7) mixture (100 ml) and the solution was kept at 5° C. overnight. A colorless precipitate was collected by filtration and dried on air for 4 hours to give crude product (3.5 g, containing 15% of D-threo diastereomer). The filtrate was treated with activated carbon at heating under reflux for half hour. The activated carbon was collected by filtration and the filtrate was kept at ambient temperature for 2 hours. A colorless needles was collected by filtration and dried at 50° C. overnight to give (D-erythro)-3-(hydroxy)-2,2-difluoro-3-(2,2-dimethyldioxolan-4-yl)propionic acid sodium salt (VI e) [1.6 g; a purity by HPLC: 96.4% (3.6% of D-threo diastereomer)]. The crude product (3.5 g) was dissolved at heating under reflux in ethyl acetate (10 ml) and the solution was kept at ambient temperature overnight. A colorless precipitate was collected by filtration, washed with diethyl ether-ethyl acetate (2:1) mixture and dried at 50° C. overnight to give an additional portion of (D-erythro)-3-(hydroxy)-2,2-difluoro-3-(2,2-dimethyldioxolan-4-yl)propionic acid sodium salt (VI e) [1.9 g, a purity by HPLC: 96.5% (3.5% of D-threo diastereomer)]. Total yield: 3.5 g (38%). This product (3.5 g) was recrystallized from ethyl acetate (10 ml) to give a pure (D-erythro)-3-(hydroxy)-2,2-difluoro-3-(2,2-dimethyldioxolan-4-yl)propionic acid sodium salt (VI e); yield: 2.9 g (31%); a purity by HPLC: 98.5% (1.5% of D-threo diastereomer); mp 117-120° C.; [α]_{D}²⁵+17.07° [c=1.01, acetonitrile (92%)-water (8%)]. ¹H NMR (DMSO-d₆): δ=1.26, 1.32 [d, 6H, C(CH₃)₂], 3.88 (m, 2H, 5-CH₂), 4.07 (t, 1H, 4-CH), 4.17 (m, 1H, 3-CH), 6.02 (s, 1H, 3-OH). ¹³C NMR (D₂O): δ=25.7, 26.4 [C(CH₃)₂], 64.3 (C-5), 70.4 (C-3, J_{C—F}=22.9, 25.0 Hz), 74.4 (C-4), 108.0 [C(CH₃)₂], 116.02 (C-2, J_{C—F}=264, 264 Hz), 169.6 (C-1, J_{C—F}=25.0, 25.0 Hz). ¹⁹F NMR indicated that mainly the one fluorine containing product was present.

### Example 9

This example describes the preparation of 2-deoxy-2,2-difluoro-D-erythro-pentofuranose-1-ulose (ribo lactone) 4—Process A.

A mixture of D-erythro-3-(hydroxy)-2,2-difluoro-3-(2,2-dimethyl-dioxolan-4-yl)propionic acid dicyclohexylammonium salt [having a purity of 99.0%, and containing 0.04% of the D-threo isomer (by HPLC), 10 g, 0.024 mol], trifluoroacetic acid (4.2 g, 2.8 ml, 0.036 mol, 1.5 equiv.), water (5 ml) and acetonitrile (100 ml) was refluxed at about 78° C. for 3 hours. The solvent (about 50 ml) was then distilled off and toluene (15 ml) was added. An azeotropic mixture (about 15 ml) was distilled off and toluene (15 ml) was again added. The procedure was repeated until the pot temperature reached 95-100° C. The solvents were then removed to dryness under reduced pressure to obtain a colorless semisolid product. Diethyl ether (80 ml) was added to the product and the mixture was stirred at ambient temperature for half an hour at 5° C. for 2 hours. The colorless crystals of trifluoroacetic acid dicyclohexyl-ammonium salt (7.0 g after drying at 50° C. overnight; 96.7% yield) were collected by filtration and the solvent was removed to dryness under reduced pressure to give 4.4 g of 2-deoxy-2,2-difluoro-D-erythro-pentofuranose-1-ulose 4 as an oil in 99.9% yield, having a purity of 93.2% by GC.

### Example 10

This example describes the preparation of 2-deoxy-2,2-difluoro-D-erythro-pentofuranose-1-ulose (ribo lactone) 4—Process B.

A mixture of D-erythro-3-(hydroxy)-2,2-difluoro-3-(2,2-dimethyl-dioxolan-4-yl)propionic acid dicyclohexylammonium salt [having a purity of 99.0%, and containing 0.45% of the D-threo isomer (by HPLC), 10 g, 0.024 mol], trifluoroacetic acid (2.94 g, 2.0 ml, 0.0257 mol, 1.05 equiv.), water (10 ml) and acetonitrile (100 ml) was refluxed at about 78° C. for 10 hours. The solvent (about 50 ml) was then distilled off and toluene (15 ml) was added. An azeotropic mixture (about 15 ml) was distilled off and toluene (15 ml) was again added. The procedure was repeated until the pot temperature reached 95-100° C. The solvents were then removed to dryness under reduced pressure to obtain a colorless semisolid product. Ethyl acetate (40 ml) was added to the product and the mixture was cooled at 5° C. overnight. The colorless crystals of trifluoroacetic acid dicyclohexyl-ammonium salt were collected by filtration and washed with cold ethyl acetate (2×10 ml). The filtrate was treated with Celite (2 g) at ambient temperature for two hours and the Celite was collected by filtration and washed with ethyl acetate (10 ml). The solvent was removed to dryness under reduced pressure to give 4.3 g of 2-deoxy-2,2-difluoro-D-erythro-pentofuranose-1-ulose 4 as an oil in 99.9% yield, having a purity of 88.1% (by GC).

### Example 11

This example describes the preparation of 2-deoxy-2,2-difluoro-D-erythro-pentofuranose-1-ulose (ribo lactone) 4—Process C.

A mixture of (D-erythro)-3-(hydroxy)-2,2-difluoro-3-(2,2-dimethyl-dioxolan-4-yl)propionic acid dicyclohexylammonium salt [having a purity of 99.0% (by HPLC) and containing 0.45% of D-threo isomer, 10 g, 0.0245 mol], water (5 ml), acetonitrile (100 ml) and sulfuric acid (1.2 ml, ˜98% purity, 0.9 mol) was refluxed at 78° C. for 3 hours. The solvent (about 50 ml) was then distilled off from the reaction mixture and toluene (75 ml) was added. An azeotropic mixture (about 75 ml) was distilled until the pot temperature reached 95-100° C. The solvents were then removed to dryness under reduced pressure to obtain a colorless semisolid product. Diethyl ether (70 ml) was added to the product and the mixture was cooled to 5° C. overnight. The colorless crystals of dicyclohexylamine sulfates were collected by filtration and washed with cold diethyl ether (4×10 ml). The solvent was removed to dryness under reduced pressure and the residue was dried at 50° C. overnight to give 4.12 g of 2-deoxy-2,2-difluoro-D-erythro-pentofuranose-1-ulose as a thick oil in 99.9% yield, having a purity of 87.6% by (GC).

### Example 12

This example describes the preparation of 2-deoxy-2,2-difluoro-D-erythro-pentofuranose-1-ulose (ribo lactone) 4—Process D.

A mixture of (D-erythro)-3-(hydroxy)-2,2-difluoro-3-(2,2-dimethyl-dioxolan-4-yl)propionic acid dicyclohexylammonium salt [having a purity of 99.0% (by HPLC), and containing 0.45% of D-threo isomer, 10 g, 0.0245 mol], water (10 ml), acetonitrile (100 ml) and sulfuric acid (1.2 ml, 98% purity, 0.9 mol) was refluxed at 78° C. for 3 hours. The solvent (about 50 ml) was then distilled off and toluene (75 ml) was added. An azeotropic mixture (about 75 ml) was distilled until the pot temperature reached 95-100° C. The solvents were then removed to dryness under reduced pressure to obtain a colorless semisolid product. Ethyl acetate (50 ml) was added to the product and the mixture was cooled at 5° C. for 2 hours. The colorless crystals of dicyclohexylamine sulfates were collected by filtration and washed with cold ethyl acetate (3×10 ml). The filtrate was washed with brine (12 ml) and the solid sodium chloride was filtered off. The organic phase was collected and dried over MgSO₄. The solvent was removed to dryness under reduced pressure and the residue was dried at 50° C. overnight to give 3.73 g of 2-deoxy-2,2-difluoro-D-erythro-pentofuranose-1-ulose as a colorless thick oil in 90.5% yield, having a purity of 93.4% (by GC).

### Example 13

This example describes the preparation of 2-deoxy-2,2-difluoro-D-erythro-pentofuranose-1-ulose (ribo lactone) 4—Process E.

A mixture of (D-erythro)-3-(hydroxy)-2,2-difluoro-3-(2,2-dimethyldioxolan-4-yl)propionic acid dicyclohexylammonium salt [having a purity of 99.0% (by HPLC), and containing 0.45% of D-threo isomer, 10 g, 0.0245 mol], water (5 ml), acetonitrile (100 ml) and sulfuric acid (1.2 ml, ˜98% purity, 0.9 mol) was refluxed at 78° C. for 3 hours. The solvent (about 80 ml) was then distilled from the reaction mixture and toluene (80 ml) was added. The mixture was dried by an azeotropic distillation with Dean-Stark trap for 2 hours. The mixture was cooled under nitrogen to ambient temperature and kept at 5° C. overnight. The colorless crystals of dicyclohexylamine sulfate were then collected by filtration and washed with cold ethyl acetate (3×10 ml). The solvents were removed to dryness under reduced pressure at 60° C. to yield 5.2 g of the crude 3,5-dihydroxy ribo lactone. The crude lactone was distillated in vacuo (at 2 mBar) to obtain 3.4 g of the pure product as a colorless thick oil in 82.5% yield, having a purity of 98.69% (by GC); [α]_{D}^{20.8}+53.79° (c=1.02, acetonitrile). GC/MS (CI): m/z=169.0 [M+H]⁺.

### Example 14

This example describes the preparation of 2-deoxy-2,2-difluoro-D-threo-pentofuranose-1-ulose (xylo lactone) 4B.

A mixture of (D-threo)-3-(hydroxy)-2,2-difluoro-3-(2,2-dimethyldioxolan-4-yl)propionic acid dicyclohexylammonium salt [having a purity of 99.0% (by HPLC), containing 1.8% of D-erythro isomer, 10 g, 0.0245 mol], water (5 ml), acetonitrile (100 ml) and sulfuric acid (1.2 ml, ˜98% purity, 0.9 mol) was refluxed at 78° C. for 3 hours. The solvent (about 70 ml) was then distilled from the reaction mixture and toluene (70 ml) was added. The mixture was dried by an azeotropic distillation with Dean-Stark trap for 2 hours. The mixture was cooled under nitrogen to ambient temperature and kept at 5° C. overnight. The colorless crystals of dicyclohexylamine sulfate were then collected by filtration and washed with cold ethyl acetate (3×10 ml). The solvents were removed to dryness under reduced pressure at 60° C. to yield a crude 3,5-dihydroxy xylo lactone (5.2 g). The crude lactone was dissolved in ethyl acetate (50 ml) and treated with brine (12 ml) and the solid sodium chloride was filtered off. The organic phase was collected and dried over MgSO₄. The solvent was removed to dryness under reduced pressure and the residue was dried at 50° C. overnight to give 3.7 g of 2-deoxy-2,2-difluoro-D-threo-pentofuranose-1-ulose as a colorless thick oil in 89.9% yield, having a purity of 96.1% (by GC), containing 3.01% of ribo lactone; [α]_{D}^{20.8}+13.66° (c=0.98, acetonitrile). GC/MS (CI): m/z=169.0 [M+H]⁺.

### Example 15

This example describes the preparation of 2-deoxy-2,2-difluoro-D-erythro-pentofuranos-1-ulose-3,5-dicinnamate.

3,5-dihydroxy-2,2-difluoro-D-erythro-pentofuranos-1-ulose, obtained by Method C, (4.15 g, 0.0245 mol) was dissolved under nitrogen at 60° C. in dry ethyl acetate (70 ml). Anhydrous pyridine (7.9 ml, 0.098 mol, 4.0 equiv.) and 4-(dimethyl-amino)pyridine (DMAP) (0.73 g, 0.0061 mol, 0.25 equiv.) were added to the solution. Then, cinnamoyl chloride (8.57 g, 0.051 mol, 2.1 equiv.) in ethyl acetate (20 ml) was added drop-wise to the mixture at ambient temperature for 10 minutes and the reaction mixture was heated at 60° C. for 6 hours. The mixture was then cooled to 0-5° C. and pyridinium hydrochloride was collected by filtration and washed with cold ethyl acetate (3×10 ml). The filtrate was cooled at 0° C. for 2 hours and an additional portion of pyridinium hydrochloride was collected by filtration. The solvent was removed to dryness under reduced pressure to obtain a colorless semisolid crude product. The crude product was dissolved in ethyl acetate (30 ml), and hexane (60 ml) was added drop-wise to the solution. The mixture was stirred at ambient temperature for 2 hours and a colorless precipitate was collected by filtration, washed with a 1:2 mixture of ethyl acetate:n-hexane (3×10 ml) and dried at 50° C. overnight to give 8.0 g of 2-deoxy-2,2-difluoro-D-erythro-pentofuranos-1-ulose-3,5-dicinnamate, having a purity of 98.7% (by HPLC). The solvents were removed to dryness under reduced pressure and the residue was slurried in toluene (15 ml) at ambient temperature for 4 hours. A colorless precipitate was then collected by filtration, washed with a 1:2 mixture of ethyl acetate:n-hexane (3×3 ml) and dried at 50° C. overnight to yield an additional 0.8 g of the product, having a purity of 98.2% (by HPLC). Total yield was 8.8 g, which is 83.8% from dicyclohexylammonium salt. Overall yield [from the compound of formula 3A: 53.6%; mp 130.0-131.0° C., [α]_{D}^{20.8}+114.0° (c=1.0, acetonitrile). ¹H NMR (DMSO-d₆): δ=4.65 (m, 2H, H-5), 5.21 (q, 1H, H-4), 5.98 (sex, 1H, H-3), 6.65 (d, J=160 Hz, 1H, CH═), 6.78 (d, J=160 Hz, 1H, CH═), 7.42 (m, 6Hₐᵣₒₘ), 7.68, 7.75 (2m, 1H in CH═ and 4 Hₐᵣₒₘ), 7.85 (d, J=160 Hz, 1H, CH═). ¹³C NMR (DMSO-d₆): δ=65.41 (C-5), 68.74 (C-3, J_{C—F}=26.8, 30.0 Hz), 77.72 (C-4 J_{C—F}=5.7 Hz), 111.99 (C-2, J_{C—F}=250, 264 Hz), 115.67 (CH═), 117.00 (CH═), 128.49, 128.80, 128.99, 129.06, 130.78, 131.18, 133.60, 133.78 (Cₐᵣₒₘ), 145.67 (CH═), 147.52 (CH═), 163.07 (C-1, J_{C—F}=30, 32 Hz), 164.79 [OC(O)CH═CHPh), 165.62 [OC(O)CH═CHPh]. GC/MS (CI): m/z=429.2 [M+H]⁺.

### Example 16

This example describes the preparation of 2-deoxy-2,2-difluoro-D-erythro-pentofuranos-1-ulose-3,5-dibenzoate.

3,5-dihydroxy-2,2-difluoro-D-erythro-pentofuranos-1-ulose, obtained by Process A, (4.3 g, 0.0245 mol) was dissolved under nitrogen at 60° C. in dry ethyl acetate (70 ml). Anhydrous pyridine (7.9 ml, 0.098 mol, 4.0 equiv.) and 4-(dimethyl-amino)pyridine (DMAP) (0.73 g, 0.0061 mol, 0.25 equiv.) were added to the solution. Then, benzoyl chloride (7.2 g, 0.051 mol, 2.1 equiv.) in ethyl acetate (20 ml) was added drop-wise to the mixture at 60° C. for 15 minutes and the reaction mixture was heated at 60-65° C. for 6 hours. The mixture was then cooled to 0-5° C. and pyridinium hydrochloride was collected by filtration and washed with cold ethyl acetate (3×10 ml). The filtrate was cooled at 0° C. for 2 hours and an additional portion of pyridinium hydrochloride was collected by filtration. The solvent was removed to dryness under reduced pressure to obtain a colorless semisolid crude product. The crude product was dissolved in dry toluene (20 ml) under heating and the mixture was cooled to ambient temperature and kept at 5-10° C. for 3 hours. A colorless precipitate was collected by filtration, washed with a mixture of 1:2 toluene:n-hexane (3×10 ml) and dried at 50° C. overnight to give 6.5 g of 2-Deoxy-2,2-difluoro-D-erythro-pentofuranos-1-ulose-3,5-dibenzoate, having a purity of 99.72% (by HPLC). The solvents were removed to dryness under reduced pressure and the thus obtained residue was slurried in toluene (10 ml) at ambient temperature for 4 hours. A colorless precipitate was then collected by filtration, washed with a 1:2 toluene:n-hexane mixture (3×5 ml) and dried at 50° C. overnight to yield an additional 0.7 g of the product, having a purity of 99.12% (by HPLC). Total yield: 7.2 g, 78.1% from the dicyclohexylammonium salt. Overall yield [from the compound of formula (IV)]: 40.1%; mp 120-121° C., [α]_{D}^{20.5}+69.67° (c=1.0, acetonitrile). ¹H NMR (DMSO-d₆): δ=4.81 (m, 2H, H-5), 5.43 (q, 1H, H-4), 6.12 (m, 1H, H-3), 7.45-7.78 (m, 6Hₐᵣₒₘ), 7.97 (m, 2Hₐᵣₒₘ), 8.08 (m, 2Hₐᵣₒₘ). ¹³C NMR (DMSO-d₆): δ=63.04 (C-5), 69.23 (C-3, J_{C—F}=26.5, 30.0 Hz), 77.72 (C-4, J_{C—F}=5.6 Hz), 111.95 (C-2, J_{C—F}=258, 260 Hz), 127.7, 128.75, 128.88, 128.96, 129.30, 129.80, 133.69, 134.40 (Cₐᵣₒₘ), 163.04 (C-1, J_{C—F}=32, 34 Hz), 164.36 [OC(O)Ph], 165.17 [OC(O)Ph]. GC/MS (CI): m/z=[M+H]⁺.

### Example 17

This example describes the preparation of 2-deoxy-2,2-difluoro-D-erythro-pentofuranos-1-ulose-3,5-di(4-chlorobenzoate).

3,5-dihydroxy-2,2-difluoro-D-erythro-pentofuranos-1-ulose, obtained by Process B, (4.3 g, 0.0245 mol) was dissolved under nitrogen at 60° C. in dry ethyl acetate (70 ml). Anhydrous pyridine (7.9 ml, 0.098 mol, 4.0 equiv.) and 4-(dimethylamino)pyridine (DMAP) (0.73 g, 0.0061 mol, 0.25 equiv.) were added to the solution. Then, 4-chlorobenzoyl chloride (8.9 g, 0.051 mol, 2.1 equiv.) in ethyl acetate (20 ml) was added drop-wise to the mixture at ambient temperature for 10 minutes and the reaction mixture was heated at 60-65° C. for 6 hours. The mixture was then cooled to 0-5° C. and pyridinium hydrochloride was collected by filtration and washed with cold ethyl acetate (3×10 ml). The filtrate was cooled at 0° C. for 2 hours and an additional portion of pyridinium hydrochloride was collected by filtration. The solvent was removed to dryness under reduced pressure at 60° C. to obtain a semisolid crude product. Toluene (50 ml) was added to the crude product and mixing was maintained for a while, after which time the toluene was removed to dryness under reduced pressure. This procedure was repeated twice to remove the traces of pyridine from the crude product and a crude solid was obtained. The crude solid was dissolved under heating in dry toluene (20 ml). The mixture was cooled to ambient temperature for 2 hours and a precipitate of 4-chlorobenzoic acid was collected by filtration. n-hexane (20 ml) was added to the filtrate and the mixture was kept at 5° C. overnight. A colorless precipitate was collected by filtration, washed with a 1:2 mixture of toluene:n-hexane (3×10 ml) and dried at 50° C. overnight to give 5.9 g of 2-Deoxy-2,2-difluoro-D-erythro-pentofuranos-1-ulose-3,5-di(4-chlorobenzoate, having a purity of 99.33% (by HPLC) in 54.1% yield. Overall yield [from the compound of formula (IV)]: 35.9%; mp 101-103° C., [α]_{D}^{20.5}+75.93° (c=0.99, acetonitrile). ¹H NMR (DMSO-d₆): δ=4.79 (m, 2H, H-5), 5.41 (m, 1H, H-4), 6.08 (m, 1H, H-3), 7.58 (d, 2Hₐᵣₒₘ), 7.65 (d, 2Hₐᵣₒₘ), 7.96 (d, 2Hₐᵣₒₘ), 8.05 (d, 2Hₐᵣₒₘ). ¹³C NMR (DMSO-d₆): δ=63.16 (C-5), 69.25 (C-3, J_{C—F}=25, 28 Hz), 77.48 (C-4, J_{C—F}=5.7 Hz), 111.84 (C-2, J_{C—F}=258, 260 Hz), 126.55, 127.70, 128.97, 129.15, 131.12, 131.60, 138.72, 139.43 (Cₐᵣₒₘ), 162.88 (C-1, J_{C—F}=32, 34 Hz), 163.54 [OC(O)C₆H₄Cl-4], 164.36 [OC(O)C₆H₄Cl-4]. GC/MS (CI): m/z=[M+H]⁺.

### Example 18

This example describes the preparation of 2-deoxy-2,2-difluoro-D-erythro-pentofuranos-1-ulose-3,5-bis(penylacetate).

3,5-dihydroxy-2,2-difluoro-D-erythro-pentofuranos-1-ulose, obtained by Method B, (4.15 g, 0.0245 mol) was dissolved under nitrogen at 60° C. in dry ethyl acetate (70 ml). Anhydrous pyridine (7.9 ml, 0.098 mol, 4.0 equiv.) and 4-(dimethyl-amino)pyridine (DMAP) (0.73 g, 0.0061 mol, 0.25 equiv.) were added to the solution. Then, phenylacetyl chloride (7.9 g, 0.051 mol, 2.1 equiv.) in ethyl acetate (20 ml) was added drop-wise to the mixture at ambient temperature for 10 minutes and the reaction mixture was heated at 60-65° C. for 6 hours and kept at ambient temperature overnight. The mixture was then cooled to 0-5° C. and pyridinium hydrochloride was collected by filtration and washed with cold ethyl acetate (3×10 ml). The filtrate was cooled at 0° C. for 2 hours and an additional portion of pyridinium hydrochloride was collected by filtration. The ethyl acetate was removed to dryness under reduced pressure from the filtrate to obtain a crude product as thick oil. Toluene (50 ml) was added to the crude product while maintaining mixing, after which time the toluene was removed to dryness under reduced pressure. This procedure was repeated twice to remove the traces of pyridine from the crude product. The crude product was dissolved in ethyl acetate (50 ml). The solution was washed with 5% NaHCO₃ (3×30 ml) and brine (2×20 ml) and dried over MgSO₄. The solvent (30 ml) was distilled off and n-hexane (60 ml) was added to the thus obtained residue. The mixture was kept at −20° C. overnight. The mixture, which consisted of two layers, was separated and the liquid lower phase was collected and dried under reduced pressure at 50° C. overnight to give 9.2 g of 2-deoxy-2,2-difluoro-D-erythro-pentofuranos-1-ulose-3,5-bis(phenylacetate) as an oil in 92.9% yield. Overall yield [from the compound of formula 3A]: 59.5%; [α]_{D}^{20.5}+° (c=1.0, acetonitrile). ¹H NMR (DMSO-d₆): δ=3.69 (s, 2H, CH₂Ph), 3.84 (s, 2H, CH₂Ph), 4.49 (octet, 2H, H-5), 5.03 (sextet, 1H, H-4), 5.76 (sextet, 1H, H-3), 7.23-7.35 (m, 10Hₐᵣₒₘ). ¹³C NMR (DMSO-d₆): δ=39.50 (CH₂Ph), 40.02 (CH₂Ph), 62.58 (C-5), 68.76 (C-3, J_{C—F}=25, 28 Hz), 77.74 (C-4, J_{C—F}=5.6 Hz), 111.85 (C-2, J_{C—F}=258, 260 Hz), 127.07, 127.26, 128.50, 128.54, 129.23, 129.49, 133.36 and 133.96 (Cₐᵣₒₘ), 162.99 (C-1, J_{C—F}=32, 34 Hz), 170.21 [OC(O)CH₂Ph], 170.79 [OC(O)CH₂Ph]. GC/MS (CI): m/z=[M+H]⁺.

### Example 19

This example describes the preparation of 2-deoxy-2,2-difluoro-D-derythro-pentofuranos-1-ulose-3,5-diacetate.

3,5-dihydroxy-2,2-difluoro-D-erythro-pentofuranos-1-ulose, obtained by Method B, (4.3 g, 0.0245 mol) was dissolved under nitrogen at 60° C. in dry ethyl acetate (70 ml). Anhydrous pyridine (7.9 ml, 0.098 mol, 4.0 equiv.) and 4-(dimethyl-amino)pyridine (DMAP) (0.73 g, 0.0061 mol, 0.25 equiv.) were added to the solution. Then, acetyl chloride (3.85 ml, 0.051 mol, 2.1 equiv.) in ethyl acetate (20 ml) was added drop-wise to the mixture at ambient temperature for 10 minutes and the reaction mixture was heated at 60-65° C. for 6 hours and kept at ambient temperature overnight. The mixture was then cooled to 0-5° C. and pyridinium hydrochloride was collected by filtration and washed with cold ethyl acetate (3×10 ml). The filtrate was cooled at 0° C. for 2 hours and an additional portion of pyridinium hydrochloride was collected by filtration. The ethyl acetate was removed to dryness under reduced pressure to obtain a crude product as an oil. Toluene (50 ml) was added to the crude product while maintaining mixing, after which time the toluene was removed to dryness under reduced pressure. This procedure was repeated twice to remove the traces of pyridine and acetic acid from the crude product. The crude product was dried under reduced pressure at 50° C. overnight to give 5.8 g of 2-deoxy-2,2-difluoro-D-erythro-pentofuranos-1-ulose-3,5-diacetate as an oil in 94.0% yield. Overall yield [from the compound of formula 3A]: 60.1%; [α]_{D}^{20.5}+° (c=1.0, acetonitrile). ¹H NMR (DMSO-d₆): δ=(s, 2H, CH₃), (s, 3H, CH₃), ( , 2H, H-5), ( , 1H, H-4), ( , 1H, H-3). ¹³C NMR (DMSO-d₆): δ=(CH₃), (CH₃), (C-5), (C-3, J_{C—F}=25, 28 Hz), (C-4, J_{C—F}=5.6 Hz), (C-2, J_{C—F}=258, 260 Hz), (C-1, J_{C—F}=32, 34 Hz), [OC(O)CH₃], [OC(O)CH₃]. GC/MS (CI): m/z=[M+H]⁺.

### Example 20

This example describes the preparation of 2-deoxy-2,2-difluoro-D-threo-pentofuranos-1-ulose-3,5-dicinnamate.

3,5-dihydroxy-2,2-difluoro-D-threo-pentofuranos-1-ulose (xylo lactone) (3.7 g, 0.022 mol) was dissolved under nitrogen at 60° C. in dry ethyl acetate (70 ml). Anhydrous pyridine (7.2 ml, 0.088 mol, 4.0 equiv.) and 4-(dimethylamino)pyridine (DMAP) (0.66 g, 0.0055 mol, 0.25 equiv.) were added to the solution. Then, cinnamoyl chloride (7.7 g, 0.046 mol, 2.1 equiv.) in ethyl acetate (20 ml) was added dropwise to the mixture at ambient temperature for 10 minutes and the reaction mixture was heated at 60° C. for 6 hours. The mixture was then cooled to 0-5° C. and pyridinium hydrochloride was collected by filtration and washed with cold ethyl acetate (3×10 ml). The filtrate was cooled at 0° C. for 2 hours and an additional portion of pyridinium hydrochloride was collected by filtration. The ethyl acetate was removed to dryness under reduced pressure to obtain a crude product as a colorless oil. Toluene (50 ml) was added to the crude product while maintaining mixing, after which time the toluene was removed to dryness under reduced pressure. This procedure was repeated twice to remove the traces of pyridine from the crude product. The crude product was dissolved in ethyl acetate (50 ml) and treated with 5% NaHCO₃ (3×20 ml) and brine (2×20 ml). The organic phase was dried over MgSO₄ and the ethyl acetate was removed to dryness under reduced pressure at 60° C. The mixture was stirred at ambient temperature for 2 hours and a colorless precipitate was collected by filtration, washed with a mixture of 1:2 ethyl acetate:n-hexane (3×10 ml) and dried at 50° C. overnight to give 7.3 g of 2-deoxy-2,2-difluoro-D-threo-pentofuranos-1-ulose-3,5-dicinnamate. The yield is 69.5% from the dicyclohexyl-ammonium salt (15B); mp ° C., [α]_{D}^{20.8}+(c=1.0, acetonitrile). ¹H NMR (DMSO-d₆): 6=(m, 2H, H-5), (q, 1H, H-4), (sex, 1H, H-3), (d, J=160 Hz, 1H, CH═), (d, J=160 Hz, 1H, CH═), (m, 6Hₐᵣₒₘ), (2m, 1H in CH═ and 4Hₐᵣₒₘ), (d, J=160 Hz, 1H, CH═). ¹³C NMR (DMSO-d₆): δ=(C-5), (C-3, J_{C—F}=26.8, 30.0 Hz), (C-4 J_{C—F}=5.7 Hz), (C-2, J_{C—F}=250, 264 Hz), (CH═), (CH═), (Cₐᵣₒₘ), (CH═), (CH═), (C-1, J_{C—F}=30, 32 Hz), [OC(O)CH═CHPh), [OC(O)CH═CHPh]. GC/MS (CI): m/z=429.2 [M+H]⁺.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for obtaining the D-erythro isomer from a mixture of D-erythro (3R) and D-threo (3S) isomers of a 3-(hydroxy)-2,2-difluoro-3-(2,2-dimethyldioxolan-4-yl)-propionic acid salt of formula 15: 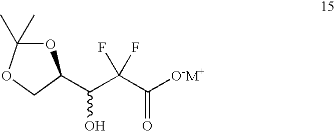
the process comprising:
dissolving a mixture of the D-erythro (3R) and D-threo (3S) isomers of formula 15 in at least one solvent, optionally at elevated temperature;
cooling the mixture sufficiently to produce crystals;
isolating the crystals;
optionally washing the crystals; and
optionally drying the crystals,
to afford the D-erythro isomer (3R)-3-(hydroxy)-2,2-difluoro-3-(2,2-dimethyldioxolan-4-yl)-propionic acid salt of formula 15A: 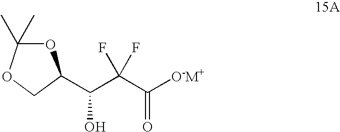
wherein M⁺ is Li⁺, Na⁺, K⁺, Ca²⁺, Ba²⁺ or R₁R₂R₃NH⁺, and R₁, R₂ and R₃ are the same or different and each is hydrogen, saturated C₁-C₁₀ alkyl, saturated C₃-C₈ cycloalkyl, unsubstituted and substituted phenyl, or unsubstituted or substituted heterocycloalkyl.

2. The process of claim 1, wherein the at least one solvent is methyl acetate, ethyl acetate, n-propyl acetate, isopropyl acetate, n-butyl acetate, isobutyl acetate, methanol, ethanol, 1-propanol, 2-propanol, acetonitrile, acetone, water, or a mixture thereof.

3. The process of claim 2, wherein the at least one solvent comprises a mixture of acetonitrile and water, or a mixture of 2-propanol and ethyl acetate.

4. The process of claim 1, wherein the D-erythro isomer of formula 15A is obtained in a purity of at least about 95% by HPLC.

5. The process of claim 1, further comprising converting the D-erythro isomer of formula 15A into gemcitabine.

6. The process of claim 5, comprising hydrolyzing the D-erythro isomer of formula 15A to produce 2-deoxy-2,2-difluoro-D-erythro-pentofuranos-1-ulose, reacting the 2-deoxy-2,2-difluoro-D-erythro-pentofuranose-1-ulose with a silylating or acylating reagent to produce a 3,5-disubstituted-2-deoxy-2,2-difluoro-D-erythro-pentofuranos-1-ulose, and converting the 3,5-disubstituted-2-deoxy-2,2-difluoro-D-erythro-pentofuranos-1-ulose into gemcitabine.
